(19) United States
(12) Patent Application Publication
(10) Pub. No.: US 2020/0170892 A1
(43) Pub. Date: Jun. 4, 2020

(54) BALANCE FUNCTIONING DENTURE TOOTH SYSTEMS CONTAINING TOUGHENED COMPOSITIONS

(71) Applicant: DENTSPLY SIRONA Inc., York, PA (US)

(72) Inventors: Benjamin Jiemin Sun, York, PA (US); Dan Ammon, York, PA (US); Brian Fritsch, Columbia, PA (US)

(73) Assignee: DENTSPLY SIRONA Inc., York, PA (US)

(21) Appl. No.: 16/782,412

(22) Filed: Feb. 5, 2020

Related U.S. Application Data
(62) Division of application No. 15/391,965, filed on Dec. 28, 2016.
(60) Provisional application No. 62/271,832, filed on Dec. 28, 2015.

(51) Int. Cl. A61K 6/887; A61C 13/00; A61K 6/64; C08F 220/18

(52) U.S. Cl. CPC A61K 6/887; A61C 13/00; A61C 5/20; C08F 220/18; A61K 6/64

(57) ABSTRACT

A dental composition that includes one or more monomers, a plurality of crosslinking agents, and an initiator.

### THE CROSS-REFERENCE TO RELATED APPLICATIONS

This patent application is division of application Ser. No. 15/391,965, filed on Dec. 28, 2016, published as US 2017/0181933, which claims the benefit of and priority to U.S. Provisional Application Ser. No. 62/271,832, filed on Dec. 28, 2015, which is herein incorporated by reference for all purposes.

### TECHNICAL FIELD

The invention relates to a method for the production of a shaped body. The invention also relates to a sintered shaped body, in particular in the form of a dental restoration, such as a dental framework, crown, partial crown, bridge, cap, veneer, abutment or pin construction.

### BACKGROUND OF THE INVENTION

Artificial teeth should exhibit certain desirable physical characteristics to be suitable for use and offer desirable benefits to patients. They should be hard for effective chewing and resistant to abrasion and chipping during use. They also should be durable and stable to solvents, foods, water, cold and hot and maintain esthetics without discoloration. In addition, they should be esthetics to mimic natural dentition with esthetically acceptable color, i.e., close to that of natural teeth. The teeth should not cause excessive wear to opposing natural or artificial teeth, crown or bridge, should not wear or deform out of occlusion, and should be capable of being bonded firmly to supportive structures. They should also be adjustable to ordinary means of physical shaping, grinding, and polishing.

Typically, artificial denture teeth are either methacrylate-based plastic teeth or ceramics-based porcelain teeth. Some composite based teeth are also available in the market. Recently, plastic teeth have largely eliminated porcelain teeth from the denture tooth market due to various advantages, such as better bond to the denture base, lighter weight, tougher, less undesirable noises during chewing and less wear to the opposing natural or artificial teeth, crown, or bridge. However, plastic teeth have the disadvantage of being more subject to wear than porcelain teeth. Of the presently available organic compositions used for the construction of artificial teeth, most are composed of acrylics, often crosslinked by polyfunctional moieties. While such compositions are commonly in use, they nonetheless possess certain drawbacks. In general, artificial teeth made of currently available acrylic compositions are not wear resistance enough and those teeth can be deformed at relatively low biting force. The deficiency in wear resistance and limited loading capability of current polymeric artificial teeth are apparent when they are against natural or ceramic teeth, crown or bridge. In addition, the use of implant therapy and overdentures becomes more popular, where a highly wear resistance artificial teeth are a must. Typically artificial teeth differ in design but are composed of the same materials that result in similar properties. There is a need for artificial teeth having better wear resistance for posterior, better fracture toughness for anterior, durability and load-bearing capability.

Various patents and tooth manufacturers have claimed the dental compositions, which offer improved wear resistance due to the use of higher crosslinked polymers and prepolymers, and composites incorporated with inorganic particles to the polymer matrix.

Erdrich et al., U.S. Pat. No. 7,368,486 disclosed dental compositions for making artificial teeth and/or their enamel or cutting area. The dental composition comprises MMA, crosslinked PMMA, splinter polymer, and a methacrylate-based pearl polymer, in which inorganic dental glass is polymerized as filler.

Rosenfeld, U.S. Pat. No. 7,189,076 discloses a method of making an artificial tooth for a denture and the tooth so made, the method including making a form tooth of a plastic tooth or an existing denture tooth, using the form tooth to make a mold form, placing a thin layer of polycarbonate incisal material in the bottom of the mold form and conforming the material to the mold form, subjecting the layer of incisal and body material to a vacuum and then to a curing light in an oxygen-free atmosphere, adding additional layers of approximately 2 mm of the incisal and body material and exposing each layer to a vacuum and light curing step as described above until the mold form is full. The tooth is then removed from the mold form and again exposed to a vacuum and light curing step.

Liu, U.S. Pat. No. 6,384,107 disclosed dental composition, product and process using silicon containing abrasion resistant material. The dental products formed are abrasion resistant and self-lubricating across their entire cross sections. Dental compositions useful for forming dental products in accordance with the invention preferably include an ethylenically unsaturated silane. The composition is formed into a dental prosthesis, such as an artificial tooth, inlay, onlay, facing, crown or bridge.

Oswald et al., EP 1,264,581 discloses a synthetic material tooth which is build up from a photopolymerizable incisor material, a photopolymerizable dentine material and, optionally, at least one other photopolymerizable material in successive intensively bonded together layers, characterized in that injection-molded or cast nipples are provided at the boundary surfaces of the layers.

Deguchi et al., U.S. Pat. No. 6,063,830 provided a dental curable composition wherein an inorganic filler treated with a silane compound is uniformly dispersed in a fine state in urethane (meth)acrylate, thereby imparting strong toughness, wear resistance, transparency and moldability to an artificial tooth. A colloidal silica has an average primary particle size of from 1 to 85 nm, with at least one silane specific compound.

Nagel et al., EP 0,677,286 discloses artificial tooth which contains polymethacrylate, barium aluminium silicate glass and microfine silica, characterized in that it essentially consists of 15-35% by weight of polymethacrylate, 35-75% by weight of barium aluminium silicate glass having a mean particle size of 0.1-5 micrometers and 5-25% by weight of silica having a mean particle size of 0.01-0.2 micrometer.

Tateosian et al., U.S. Pat. No. 4,698,373 discloses compositions hardenable by exposure to heat or electromagnetic radiation by dissolving together to form a blend form about 0% to about 50% by weight of an uncrosslinked polymer, from about 2% to about 30% of a polymerizable monomer, from about 10% to about 70% of a crosslinked polymer in the form of discrete particles having average diameters of from 0.001 micron to about 500 microns and being swollen in said solution and from about 20% to about 70% of a crosslinking agent for said monomer.

Roemer et al., U.S. Pat. Nos. 4,396,476 and 4,396,377 disclose compositions hardenable by exposure to heat or electromagnetic radiation by blending form about 0% to about 50% by weight of an uncrosslinked polymer, from about 20% to about 66% of a polymerizable monomer capable of dissolving said polymer, from about 10% to about 70% of a crosslinked polymer in the form of discrete particles having average diameters of from 0.001 micron to about 500 microns and being swollen in said monomer, and from about 0.25% to about 27% of a crosslinking agent for said monomer.

Although some artificial teeth materials described in the patents have some desirable properties, there is a need for developing new tooth material with improved wear resistance, fracture toughness, strength, and esthetics and at the same time, easy to manufacture. In general, most denture teeth breakages are related to anterior denture teeth while the wear out of denture teeth is associated with posterior denture teeth. It is desirable to have anterior teeth with superior fracture resistance while posterior teeth with superior wear resistance. At the same time, both anterior and posterior teeth provided improved tooth bonding strength to acrylic denture base.

The object of this invention is to provide a unique set of denture teeth to meet the need of different physical property requirement for anterior and posterior teeth as well as develop denture tooth compositions to meet this need, which are useful in the construction of artificial teeth, their dentins and enamels. These compositions lead to products having improved wear resistance, strength, bond strength and modulus for posterior denture teeth and superior fracture toughness and bonding strength for anterior denture teeth.

### SUMMARY OF INVENTION

A unique denture tooth system was developed, where anterior teeth and posterior teeth use different materials to meet the need for different performance. In general, several different high wear resistant compositions and high toughness compositions of this invention are useful for the formation, construction of different layers and types of artificial denture teeth, such as artificial anterior and posterior teeth, their enamels and dentins with different compositions.

It is the objective of this invention to provide the enamels of posterior teeth with at least 10% difference in wear resistance than the enamels of anterior teeth. It is the objective of the invention to provide the enamels of anterior teeth with at least 10% difference in toughness than the enamels of posterior teeth. It is the objective of the invention to provide the enamels of posterior teeth with at least 10% difference in toughness than the dentin of posterior teeth. It is also the objective of the invention to provide new denture teeth with improved tooth bonding strength to denture base. The better bonding strength provides tougher denture teeth due to the much improved bonding interfaces, which offers strengthening/synergistic effect for stronger and more durable denture teeth.

This invention provides a unique denture tooth system where anterior teeth and posterior teeth having different materials to meet the need for different performance. Toughened, high strength and high wear resistant polymerizable compositions were developed to meet this need, which are useful for a wide range of applications. Particular utility is found in the dental field where such compositions are highly suitable for the formation and construction of artificial teeth, their enamels, dentins and for other dental and prosthetic uses, such as denture base, denture baseplates, denture liners, denture repair, splints, orthodontic appliances, custom trays, veneer, crown and bridge, repair for natural teeth, and teeth restorative fillings, etc. Specifically, the invention relates to denture tooth system where anterior teeth and posterior teeth using different materials to meet the need of high fracture toughness, excellent bonding to denture base and high strength for anterior teeth and high wear resistance, good bonding to denture base and high strength for posterior teeth. The invention also relates to polymeric compositions comprising polymers, crosslinked polymers, monomers, and urethane based multifunctional crosslinking monomers or oligomers, especially urethanes (meth)acrylates, for said monomers which form precursor blends. These precursor blends are capable of being formed or molded and caused to polymerize to provide articles possessing superior wear resistance, surprising good toughness due to the use of urethane (meth)acrylates as crosslinker, desirable physical and physiochemical properties, such as high flexural strength. Furthermore, excellent molding processability is useful in the production of an artificial tooth material. This invention also relates to materials for an artificial tooth produced therefrom and materials for the tooth having excellent moldability. The invention also relates to compression molding, 3D printing, CAD/CAM processed and transfer molding of different polymerizable materials to form prosthetic teeth with several layers consisting of different polymerizable materials. The design of several layers in artificial teeth with different materials is similar to natural dentition consisting of multiple layers. Different layers of materials showed different performance attributes.

In another aspect, the present invention contemplates a dental composition comprising: (i) about 40 to about 95 wt. % one or more monomers selected from the group of methyl methacrylate; methyl acrylate; ethyl methacrylate; isobutyl methacrylate; cyclohexylmethacrylate; isobornyl methacrylate; isobornyl acrylate; allyl methacrylate; and mixtures thereof; (ii) about 0 to about 15 wt. % a first crosslinking agent selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof; (iii) about 5 to about 20 wt. % a second crosslinking agent selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof; iv) about 5 to about 40 wt. % of a third crosslinking agent selected from the group consisting of a reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane and 2-hydroxyethyl methacrylate, 2-hydroxy-3-phenoxypropyl acrylate, 2-hydroxyethyl acrylate, a reaction product of 1,3-bis(isocyanatomethyl)cyclohexane and 2-hydroxyethyl methacrylate, 2-hydroxy-3-phenoxypropyl acrylate, 2-hydroxyethyl acrylate; and mixtures thereof; (v) about 0 to about 20 wt. % of a forth crosslinking agent selected from the group consisting of a reaction product of trimethyl 1,6-diisocyanatohexane and bisphenol A propoxylate and 2-hydroxyethyl methacrylate; a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof; and (vi) about 0 to about 10 wt % an initiator.

In another aspect, the present invention contemplates an artificial tooth comprising a composition including: a) about 35 to about 60% by weight liquid component including: (i) about 60 to about 95 wt. % one or more monomers selected from the group of methyl methacrylate, methyl acrylate, ethyl methacrylate, isobutyl methacrylate, cyclohexylmethacrylate, isobornyl methacrylate, isobornyl acrylate, allyl methacrylate and mixtures thereof; (ii) about 0 to about 15 wt. % a first crosslinking agents selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof; (iii) about 0.5 to about 20 wt. % a second crosslinking agents selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof; (iv) about 0.5 to about 15 wt. % of a third crosslinking agent selected from the group consisting of a reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane and 2-hydroxyethyl methacrylate, 2-hydroxy-3-phenoxypropyl acrylate, 2-hydroxyethyl acrylate, a reaction product of 1,3-bis(isocyanatomethyl)cyclohexane and 2-hydroxyethyl methacrylate, 2-hydroxy-3-phenoxypropyl acrylate, 2-hydroxyethyl acrylate; and mixtures thereof; (v) about 0 to about 20 wt. % of a forth crosslinking agent selected from the group consisting of a reaction product of trimethyl 1,6-diisocyanatohexane and bisphenol A propoxylate and 2-hydroxyethyl methacrylate; a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof; (vi) about 0 to about 10 wt % an initiator that includes a peroxide, more particularly benzoyl peroxide; and b) about 30 to about 65% by weight of particulate material.

In another aspect, the present invention contemplates an artificial tooth comprising a composition including: a) about 35 to about 60% by weight liquid component including: (i) about 40 to about 95 wt. % one or more monomers selected from the group of methyl methacrylate, methyl acrylate, ethyl methacrylate, isobutyl methacrylate, cyclohexylmethacrylate, isobornyl methacrylate, isobornyl acrylate, allyl methacrylate and mixtures thereof; (ii) about 0 to about 15 wt. % a first crosslinking agents selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof; (iii) about 0 to about 20 wt. % a second crosslinking agents selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof; (iv) about 0 to about 40 wt. % of a third crosslinking agent selected from the group consisting of a reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane and 2-hydroxyethyl methacrylate, 2-hydroxy-3-phenoxypropyl acrylate, 2-hydroxyethyl acrylate, a reaction product of 1,3-bis(isocyanatomethyl)cyclohexane and 2-hydroxyethyl methacrylate, 2-hydroxy-3-phenoxypropyl acrylate, 2-hydroxyethyl acrylate; and mixtures thereof; (v) about 0 to about 20 wt. % of a forth crosslinking agent selected from the group consisting of a reaction product of trimethyl 1,6-diisocyanatohexane and bisphenol A propoxylate and 2-hydroxyethyl methacrylate; a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof; and (vi) about 0 to about 10 wt % an initiator that includes a peroxide, more particularly benzoyl peroxide; and b) about 30 to about 65% by weight of particulate material including: (i) a poly(methyl methacrylate) based component, and (ii) a modified Poly(methyl methacrylate).

In another aspect, the present invention contemplates an artificial tooth comprising a composition including: a) about 1 to about 80% by weight of particulate material including: (i) about 40 to about 60 wt. % methyl methacrylate; (ii) about 0 to about 15 wt. % ethylene glycol dimethacrylate; (iii) about 10 to about 20 wt. % 2,2-bis(4-methacryloxyphenyl)propane; (iv) about 15 to about 40 wt. % of a reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate; (v) about 0 to about 10 wt. % of a reaction product of trimethyl 1,6-diisocyanatohexane and bisphenol A propoxylate and 2-hydroxyethyl methacrylate; and (vi) about 0 to about 10 wt % benzoyl peroxide; and b) about 30 to about 65% by weight of particulate material.

In yet another aspect, any of the aspects of the present invention may be further characterized by one or any combination of the following features: the artificial tooth has a wear loss ranging from about 0.015 to about 0.080 (Volume Loss: 37° C., mm³); the artificial tooth has a wear loss ranging from about 0.035 to about 0.070 (Volume Loss: 37° C., mm³); the artificial tooth has a flexural strength ranging from about 125 to about 155 (MPa); the artificial tooth has a flexural strength ranging from about 135 to about 145 MPa); the artificial tooth has a modulus ranging from about 2750 to about 3750 (MPa); the artificial tooth has a modulus ranging from about 3000 to about 3500 MPa (MPa); the artificial tooth has a fracture toughness ranging from about 0.85 to about 1.85 (MPa m^{1/2}); the artificial tooth has a fracture toughness ranging from about 1.1 to about 1.6 (MPa m^{1/2}); the artificial tooth has a fracture toughest ranging from about 1.6 to about 2.7 (MPa m^{1/2}); the artificial tooth has a fracture toughest ranging from about 1.8 to about 2.5 (MPa m^{1/2}); the artificial tooth has a wear resistance ranging from about 0.045 to about 0.14, (Volume Loss: 37° C., mm³); the artificial tooth has a wear resistance ranging from about 0.06 to about 0.125, (Volume Loss: 37° C., mm³); wherein the composition further includes about 30 to about 65% by weight of particulate material; wherein the particulate material includes one or more PMMA polymers, one or more crosslinked/modified PMMA polymers, and/or mixtures thereof; wherein the one or more PMMA polymers includes Methyl Methacrylate Polymer; wherein the one or more crosslinked/modified PMMA polymers includes a Methyl Methacrylate/Ethylene Glycol Dimethacrylate Copolymer; wherein the composition includes a ratio of the one or more PMMA polymers to the one or more crosslinked/modified PMMA polymers ranging from about 4:1 to about 1:4; wherein the composition includes a ratio of the one or more PMMA polymers to the one or more crosslinked/modified PMMA polymers ranging from about 1:1 to about 1:3; wherein the one or more crosslinked/modified PMMA polymers includes a ratio of the one or more PMMA polymers to Ethylene Glycol Dimethacrylate ranging from about 4:1 to about 1:4; wherein the one or more crosslinked/modified PMMA polymers includes a ratio of the one or more PMMA polymers to Ethylene Glycol Dimethacrylate ranging from about 1:1 to about 1:3; or any combination thereof.

### DETAILED DESCRIPTION OF INVENTION

In accordance with a preferred form of the present invention, polymerizable dental compositions are provided which may easily and conveniently be molded by known techniques into prosthetic denture teeth possessing chemical and physical properties which are significantly improved over those of conventional prior art acrylic prosthetic teeth produced from precursor blend compositions prepared in accordance with the invention are characterized by high wear resistance for posterior teeth and high fracture toughness for anterior teeth. Specifically, a high wear resistance composition was developed for enamels of posterior teeth, a high fracture toughness composition for dentins of anterior and posterior teeth and a high bonding strength composition for neck or dentin of all denture teeth.

Furthermore, prosthetic teeth produced from compositions of the invention have excellent stain, chemical and solvent resistances. Their excellent bonding strength to acrylic denture base is superior to many premium plastic teeth in the market.

In comparison with conventional highly crosslinked acrylic teeth, the prosthetic teeth, especially enamel surfaces of posterior teeth, produced in accordance with the invention are characterized by outstanding wear resistance to reduce any wear issues associated with posterior denture teeth, excellent monomer and solvent resistance, outstanding thermal stability, improved hardness, improved shape stability due to the enhanced modulus of enamel layer and excellent hydrolytic stability. Teeth produced from the compositions of the invention exhibit excellent gloss when molded, excellent bonding to denture base due to better bondable dentin in these denture teeth. During denture fabrication, the glosses of these teeth are maintained better than that of conventional highly crosslinked acrylic teeth and standard acrylic plastic teeth, due to the higher crosslinking density, superior chemical and wear resistances of enamel layers.

In comparison with conventional highly crosslinked acrylic teeth, the prosthetic teeth, especially dentin area of anterior teeth, produced in accordance with the invention are characterized by outstanding fracture toughness to prevent any breakage issues associated with anterior denture teeth, excellent bonding ability, where denture base can better bonding to these denture teeth and offer better durability, outstanding thermal stability, improved modulus and strength.

The precursor blend is formed in accordance with the invention by combining a monomer, crosslinking agents for said monomer, such as an urethane based crosslinking agent, especially an aromatic and/or cyclic ring structure based urethane crosslinker, crosslinked polymer and an optional uncrosslinked polymer, and/or an initiator and by allowing said combination to age or mature.

In general, artificial plastic teeth are made from PMMA and modified PMMA polymers and MMA and modified MMA liquids. More particularly, the powder material may include one or more PMMA polymers (Methyl Methacrylate Polymer), one or more crosslinked/modified PMMA polymers (e.g., Methyl Methacrylate: Ethylene Glycol Dimethacrylate Copolymer) and mixtures thereof. When both included, the PMMA polymer and the crosslinked/modified PMMA polymers may be present in a ratio ranging from about 4:1 to about 1:4, preferably about 1:1 to about 1:3, and more preferably approximately about 1:2. Furthermore, the one or more crosslinked/modified PMMA polymer may include Methyl Methacrylate to Ethylene Glycol Dimethacrylate in a ratio ranging from about 4:1 to about 1:4, preferably about 1:1 to about 1:3, and more preferably approximately about 1:2.

In general, the crosslinked polymers which are useful in the practice of the invention are formed from monomers or blends of monomers together with crosslinking agents in proper proportion. Monomer compounds that can be used in the composition of this invention, include, but are not limited to, methyl methacrylate, methyl acrylate, ethyl methacrylate, isobutyl methacrylate, cyclohexylmethacrylate, isobornyl methacrylate, isobornyl acrylate, allyl methacrylate, etc.

Crosslinking agents that can be used in the composition of this invention, include, but are not limited to, di- or poly-acrylates and methacrylates such as glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate, 2,2-bis[4-(2-hydroxy-3-acryloyloxypropoxy)phenyl]propane; 2,2-bis[4-(2-hydroxy-3-methacryloyloxypropoxy)phenyl]propane (Bis-GMA); 2,2-bis[4-(acryloyloxy-ethoxy)phenyl]propane; 2,2-bis[4-(methacryloyloxy-ethoxy)phenyl]propane (or ethoxylated bisphenol A-dimethacrylate) (EBPADMA); urethane di(meth)acrylate (UDMA), diurethane dimethacrylate (DUDMA), 4,13-dioxo-3,14 dioxa-5,12-diazahexadecane-1,16-diol diacrylate; 4,13-dioxo-3,14 dioxa-5,12-diazahexadecane-1,16-diol dimethacrylate; the reaction product of trimethyl 1,6-diisocyanatohexane and bisphenol A propoxylate and 2-hydroxyethyl methacrylate (TBDMA); the reaction product of 1,6 diisocyanatohexane and 2-hydroxyethyl methacrylate modified with water (HDIDMA); the reaction product of 1,6 diisocyanatohexane and 2-hydroxyethyl acrylate modified with water (HDIDA); polyurethane dimethacrylate (PUDMA); alkoxylated pentacrythritol tetraacrylate; polycarbonate dimethacrylate (PCDMA); the bis-acrylates and bis-methacrylates of polyethylene glycols; and copolymerizable mixtures of acrylated monomers and acrylated oligomers, etc.

Preferably, urethane based diacrylate or dimethacrylate can be used to offer improved fracture toughness, especially urethane (meth)acrylates containing cyclic backbone or aromatic structures. It is found surprisingly the use of urethane (meth)acrylates containing cyclic backbone and/or aromatic structures as crosslinker to replace a part of conventional crosslinkers, such as ethylene glycol dimethacrylate (EGDMA) or bisphenol A-dimethacrylate (BPADMA) resulted in significant improvement in the fracture toughness of formed denture tooth materials. The urethane structure formed in polymer network generated a toughened structure which resulted in improved fracture toughness and flexural strength and modulus.

It has been discovered that the relative proportions of the crosslinkers of the liquid blend used in accordance with the invention are critical to the attainment of the desired properties in the final hardened or cured product produced therefrom, notably the wear resistance, bond strength, flexural properties, impact strength, fracture toughness, resistance to MMA monomer and other solvents, stain resistance, thermal stability, and hydrolytic stability, especially the balance of fracture toughness and wear resistance. Thus, it has been discovered that liquid blends containing from about 2 to 40 weight percent of the urethane based crosslinkers, from about 0 to 30 weight percent of BPADMA, from 0 to 30 weight percent of other crosslinkers, from about 30 to about 95 weight percent of polymerizable monomer, and from about 5 to about 70 weight percent of crosslinked agents for said monomer, together with minor amounts of initiator and in some cases activator for the initiator, provide liquid blends which are particularly useful in the production of enamel layers of prosthetic teeth or prosthetic teeth with properties, especially wear resistance, far superior to those of conventional acrylic systems now used in the art while maintain excellent fracture toughness. The novel feature of this system is the introduction of unique urethane crosslinkers, which enhanced the fracture toughness of cured product surprisingly.

This invention developed a denture teeth system with different performance for anterior and posterior denture teeth since anterior and posterior teeth functioned differently in oral environment and required different material properties. It is reported that the excess wear typically occurred among posterior teeth over their lifetime while the breakage issue is often associated with anterior teeth. It is the intention of this invention to develop a set of denture teeth with tougher anterior teeth and high wear resistant posterior teeth. The compositions of this invention enabled the development of the enamels of posterior teeth with at least 10% difference in wear resistance than the enamels of anterior teeth. It is more preferable that the enamels of posterior teeth with at least 15% difference in wear resistance than the enamels of anterior teeth. It is also preferable that the enamels of anterior teeth with at least 10% difference in fracture toughness than the enamels of posterior teeth. It is more preferable that the enamels of anterior teeth with at least 15% difference in fracture toughness than the enamels of posterior teeth. In addition, it is preferable that the dentins of posterior teeth with at least 10% difference in fracture toughness than the enamels of posterior teeth. It is more preferable that the dentins of posterior teeth with at least 15% difference in fracture toughness than the enamels of posterior teeth. It is most preferable that the dentins of posterior teeth with at least 20% difference in fracture toughness than the enamels of posterior teeth. It is also desirable to provide new denture teeth with improved tooth bonding strength to denture base. The tooth bonding layer material has relatively lower crosslinking density for better bonding. The better bonding strength provides tougher denture teeth due to the much improved bonding interfaces, which offers strengthening/synergistic effect for stronger and more durable denture teeth.

Liquid
Component

| | | | | |
|---|---|---|---|---|
| Monomer(s) | (35 to 95) | (35 to 95) | (40 to 95) | (60 to 95) |
| | (40 to 60) | (60 to 85) | (75 to 85) | (80 to 95) |
| First | (0 to 15) | (0 to 20) | (0 to 10) | (0 to 15) |
| Crosslinking | (0.5 to 15) | (0.5 to 15) | (0.5 to 10) | (0.5 to 15) |
| Agent | | | | |
| Second | (5 to 25) | (0.5 to 25) | (0.5 to 20) | (0 to 10) |
| Crosslinking | | | | |
| Agent | (10 to 20) | (10 to 17.5) | (5 to 15) | (0.5 to 5) |
| Third | (5 to 45) | (0 to 20) | (0 to 25) | (0 to 15) |
| Crosslinking | (15 to 40) | (0.5 to 15) | (7.5 to 17.5) | (0.5 to 10) |
| Agent | | | | |
| Forth | (0 to 20) | (0 to 20) | (0 to 20) | (0 to 20) |
| Crosslinking | (0.5 to 10) | (0.5 to 10) | (0.5 to 10) | (0.5 to 10) |
| Agent | | | | |
| Initiator | (0 to 10) | (0 to 10) | (0 to 10) | (0 to 10) |
| | (0.5 to 7.5) | (0.5 to 7.5) | (0.5 to 7.5) | (0.5 to 7.5) |
| Total | 100 | 100 | 100 | 100 |
| Mix Ratio | L:P = 50:50 | L:P = 46:54 | L:P = 46:54 | L:P = 46:54 |
| Liquid:Powder | | | | |
| (L:P) | | | | |

** Tooth Composition includes a liquid component mixture and a particulate/powder material mixture that may include a mixture of PMMA homepolymer and crosslinked PMMA polymer.

### Example 1

The benzoyl peroxide (0.5 wt %), 2,2-bis(4-methacryloxyphenyl)propane (BPADMA) (16 wt %), the reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate (22.5 wt %) and ethylene glycol dimethacrylate (13 wt %) were dissolved in the methyl methacrylate (48 wt %) at ambient temperature to form a monomer solution, then mixed with polymer powders (1:1 weight ratio of liquid to powder) to form a visibly homogeneous dough. The enamel layers of prosthetic teeth were molded from the resultant precursor blend mixture after it was aged at ambient temperature. A suitable gel-like consistency for molding prosthetic teeth was obtained after aging at ambient temperature. The resulting material has excellent wear resistance and flexural properties.

### Example 2

The benzoyl peroxide (0.5 wt %), 2,2-bis(4-methacryloxyphenyl)propane (BPADMA) (16 wt %), the reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate (22.5 wt %) and ethylene glycol dimethacrylate (7 wt %) were dissolved in the methyl methacrylate (54 wt %) at ambient temperature to form a monomer solution, then mixed with polymer powders (48:52 weight ratio of liquid to powder) to form a visibly homogeneous dough. The enamel layers of prosthetic teeth were molded from the resultant precursor blend mixture after it was aged at ambient temperature. A suitable gel-like consistency for molding prosthetic teeth was obtained after aging at ambient temperature. The resulting material has excellent wear resistance and flexural properties.

### Example 3

The benzoyl peroxide (0.5 wt %), and 2,2-bis(4-methacryloxyphenyl)propane (BPADMA) (17.3 wt %) were dissolved in the methyl methacrylate (82.2 wt %) at ambient temperature to form a monomer solution, then mixed with polymer powders (46:54 weight ratio of liquid to powder) to form a visibly homogeneous dough. The dentin layers of prosthetic teeth were molded from the resultant precursor blend mixture after it was aged at ambient temperature. A suitable gel-like consistency for molding prosthetic teeth was obtained after aging at ambient temperature. The resulting material has excellent fracture toughness and good wear resistance.

### Example 4

The benzoyl peroxide (0.5 wt %), 2,2-bis(4-methacryloxyphenyl)propane (BPADMA) (14.9 wt %), and the reaction product of 1,3-bis(isocyanatomethyl)cyclohexane and 2-hydroxyethyl methacrylate, 2-hydroxy-3-phenoxypropyl acrylate, 2-hydroxyethyl acrylate and ethylene glycol dimethacrylate (2.9 wt %) were dissolved in the methyl methacrylate (81.7 wt %) at ambient temperature to form a monomer solution, then mixed with polymer powders (46:54 weight ratio of liquid to powder) to form a visibly homogeneous dough. The dentin layers of prosthetic teeth were molded from the resultant precursor blend mixture after it was aged at ambient temperature. A suitable gel-like consistency for molding prosthetic teeth was obtained after aging at ambient temperature. The resulting material has excellent fracture toughness and good wear resistance.

### Example 5

The benzoyl peroxide (0.5 wt %), 2,2-bis(4-methacryloxyphenyl)propane (BPADMA) (8.5 wt %), and the reaction product of 1,3-bis(isocyanatomethyl)cyclohexane and 2-hydroxyethyl methacrylate, 2-hydroxy-3-phenoxypropyl acrylate, 2-hydroxyethyl acrylate (11.6 wt %) were dissolved in the methyl methacrylate (79.4 wt %) at ambient temperature to form a monomer solution, then mixed with polymer powders (46:54 weight ratio of liquid to powder) to form a visibly homogeneous dough. The dentin layers of prosthetic teeth were molded from the resultant precursor blend mixture after it was aged at ambient temperature. A suitable gel-like consistency for molding prosthetic teeth was obtained after aging at ambient temperature. The resulting material has excellent fracture toughness and good wear resistance.

### Example 6

The benzoyl peroxide (0.5 wt %), 2,2-bis(4-methacryloxyphenyl)propane (BPADMA) (4 wt %), and the reaction product of 1,3-bis(isocyanatomethyl)cyclohexane and 2-hydroxyethyl methacrylate, 2-hydroxy-3-phenoxypropyl acrylate, 2-hydroxyethyl acrylate (11.6 wt %) were dissolved in the methyl methacrylate (83.9 wt %) at ambient temperature to form a monomer solution, then mixed with polymer powders (46:54 weight ratio of liquid to powder) to form a visibly homogeneous dough. The dentin or neck layers of prosthetic teeth were molded from the resultant precursor blend mixture after it was aged at ambient temperature. A suitable gel-like consistency for molding prosthetic teeth was obtained after aging at ambient temperature. The resulting material has excellent fracture toughness and bond strength to acrylic denture base.

### Example 7

The benzoyl peroxide (0.5 wt %), 2,2-bis(4-methacryloxyphenyl)propane (BPADMA) (3 wt %), and the reaction product of 1,3-bis(isocyanatomethyl)cyclohexane and 2-hydroxyethyl methacrylate, 2-hydroxy-3-phenoxypropyl acrylate, 2-hydroxyethyl acrylate (7.5 wt %) were dissolved in the methyl methacrylate (89 wt %) at ambient temperature to form a monomer solution, then mixed with polymer powders (46:54 weight ratio of liquid to powder) to form a visibly homogeneous dough. The dentin or neck layers of prosthetic teeth were molded from the resultant precursor blend mixture after it was aged at ambient temperature. A suitable gel-like consistency for molding prosthetic teeth was obtained after aging at ambient temperature. The resulting material has excellent fracture toughness and bond strength to acrylic denture base.

### Wear Resistance Tests

Wear resistance was tested using a three-body cyclic abrasion wear machine (Leinfelder method) at 37° C. Localized wear was measured by determining volume loss in mm³ after 400,000 cycles at 50 RPM. The wear data for sample prepared from Examples 1 to 7 are listed in Table 1.

TABLE 1
Wear loss of tooth materials of this invention tested at 37° C.

| Material | Volume loss (37° C., mm³) | S.D. |
|---|---|---|
| Example 1 | 0.051 | 0.017 |
| Example 2 | 0.059 | 0.010 |
| Example 3 | 0.075 | 0.015 |
| Example 4 | 0.074 | 0.015 |
| Example 5 | 0.093 | 0.017 |
| Example 6 | 0.097 | 0.007 |
| Example 7 | 0.111 | 0.014 |

### Flexural Property Tests

Flexural Strength and Flexural Modulus of the polymerized compositions of Examples 1 to 7 were measured with crosshead speed of 1 mm/minute by using three-point bend test on Instron bending unit according to ISO. Samples (2 mm×2 mm×25 mm) from Examples 1 to 6 were molded in metal molds with the same curing cycles and post cure in 260° F. oven for two hours.

TABLE 2
Flexural strength and flexural modulus of tooth materials
of this invention tested at ambient temperature.

| Material | Flex Strength (MPa) | Modulus (MPa) |
|---|---|---|
| Example 1 | 140 (sd = 8) | 3277 (sd = 172) |
| Example 2 | 140 (sd = 7) | 3268 (sd = 162) |
| Example 3 | 131 (sd = 5) | 2875 (sd = 185) |
| Example 4 | 134 (sd = 3) | 2944 (sd = 121) |
| Example 5 | 129 (sd = 2) | 2893 (sd = 57) |
| Example 6 | 126 (sd = 7) | 3038 (sd = 139) |
| Example 7 | 127 (sd = 4) | 2989 (sd = 82) |

### Fracture Toughness Tests

Fracture toughness of the polymerized compositions of Examples 1 to 7 was measured by Instron with a crosshead speed of 0.6 mm/minute. Cylindrical short rod fracture toughness test specimens were machined and tested in accordance with ASTM E1304-97 [Standard Test Method for Plane-Strain (Chevron Notch) Fracture Toughness of Met allic Materials]. Chevron-cut samples were placed into 37° C. deionized water for 24 hours, followed by 1 hour at 23° C. deionized water prior to testing.

TABLE 3
Fracture toughness of tooth materials of this
invention tested at ambient temperature.

| Material | K_{IC} (MPa m^{1/2}) |
|---|---|
| Example 1 | 1.33 (sd = 0.23) |
| Example 2 | 1.72 (sd = 0.14) |
| Example 3 | 1.91 (sd = 0.15) |
| Example 4 | 1.99 (sd = 0.18) |
| Example 5 | 2.32 (sd = 0.13) |
| Example 6 | 2.28 (sd = 0.07) |
| Example 7 | 2.26 (sd = 0.06) |

Example 1 and 2 have the best wear resistance (about 0.015 to about 0.080, preferably about 0.035 to about 0.070, Volume Loss: 37° C., mm³) and flexural strength (about 125 to about 155, preferably about 135 to about 145 MPa) and modulus (about 2750 to about 3750, preferably, about 3000 to about 3500 MPa) but lower fracture toughness (about 0.85 to about 1.85, preferably, about 1.1 to about 1.6 MPa m^{1/2}), so they are better suited for enamels of posterior teeth. A posterior enamel formulation displays higher crosslinking density, better wear resistance and higher modulus, which can better maintain desirable occlusal details (better dimensional stability) than current commercially available plastic denture tooth and IPN denture tooth materials, are highly desirable, where bite load and chewing motion often can result in excess wear or squash off occlusal details. Example 4, 5, 6 and 7 have the highest fracture toughest (e.g., about 1.6 to about 2.7, preferably, about 1.8 to about 2.5 MPa m^{1/2}), but relatively lower wear resistance (e.g., about 0.045 to about 0.14, preferably about 0.06 to about 0.125 Volume Loss: 37° C., mm³), which are best suited for dentins (including body and neck materials). Examples 6 and 7 have lowest crosslinking density, which can provide best bonding to denture base. An anterior enamel formulation is required for a stronger and tougher denture tooth to withstand the bite force from opposing dentition or denture teeth, where the wear of denture teeth is less significant compared to posterior denture teeth. It is preferable to use materials from Example 3, 4 and 5 for enamels of anterior teeth. Compared to porcelain denture teeth, polymeric denture teeth have less resistance to creep, higher fracture toughness, better resistance to thermal shock, higher water sorption, and bond to denture base. In contrast, porcelain denture teeth show better dimensional stability and have much increased wear resistance. A higher wear resistant (about 0.015 to about 0.080, preferably about 0.035 to about 0.070, Volume Loss: 37° C., mm³) and higher modulus (about 2750 to about 3750, preferably, 3000 to about 3500 MPa) enamel layer is desirable, especially for posterior teeth, where bite load is much higher than anterior teeth. It should be understand that while the present invention has been described with respect to certain specific embodiments thereof, it should not be considered limited to such embodiments but may be used in other ways without departure from the spirit of the invention and the scope of the appended claims.

The artificial teeth composition, wherein said first resin for enamel layer has greater wear resistance than said second resin for body layer and said second resin has greater wear resistance than said third resin for neck layer.

The artificial teeth composition wherein said second and third resins for dentin layer or body and neck layers have greater fracture toughness than said first resin for enamel layer.

The artificial teeth composition, wherein said first resin for enamel layer of posterior denture teeth has greater wear resistance than said first resin for enamel layer of anterior denture teeth.

The artificial teeth composition, wherein said first resin for enamel layer of anterior denture teeth has at least 10% difference in wear resistance than said first resin for enamel layer of posterior denture teeth.

The artificial teeth composition, wherein said first resin for enamel layer of anterior denture teeth has greater fracture toughness than said first resin for enamel layer of posterior denture teeth.

The artificial teeth composition, wherein said first resin for enamel layer of anterior denture teeth has at least 10% difference in fracture toughness than said first resin for enamel layer of posterior denture teeth.

The artificial teeth composition, wherein said first resin for enamel layer of posterior denture teeth has at least 10% difference in wear resistance than said second or third resin for dentin layer of anterior and posterior denture teeth.

The artificial teeth composition, wherein said first resin for enamel layer of posterior denture teeth has at least 10% difference in fracture toughness than said second and third resins for dentin layer or body and neck layers of anterior and posterior denture teeth.

The artificial teeth composition, wherein the liquid component further includes at least one of urethane (meth)acrylate based crosslinking agent.

The artificial teeth composition, wherein the at least one of urethane (meth)acrylate based crosslinking agent includes an aromatic or cyclic backbone structure.

What is claimed is:

1. A denture tooth system having anterior and posterior teeth, wherein enamels of anterior teeth and enamels of posterior teeth have different material, wherein the material is obtained by forming or molding and causing to polymerize a dental composition, the dental composition comprising:
(i) about 40 to about 95 wt. % one or more monomers selected from the group of methyl methacrylate; methyl acrylate; ethyl methacrylate; isobutyl methacrylate; cyclohexylmethacrylate; isobornyl methacrylate; isobornyl acrylate; allyl methacrylate; and mixtures thereof;
(ii) about 0 to about 15 wt. % a first crosslinking agent selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof;
(iii) about 5 to about 20 wt. % a second crosslinking agent selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof;
(iv) about 5 to about 40 wt. % of a third crosslinking agent selected from the group consisting of a reaction product of 1,3-bis(isocyanatomethyl)cyclohexane, 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane, 2-hydroxyethyl methacrylate, 2-hydroxy-3-phenoxypropyl acrylate, 2-hydroxyethyl acrylate; and mixtures thereof;
(v) about 0 to about 20 wt. % of a fourth crosslinking agent selected from the group consisting of a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof; and
(vi) about 0 to about 10 wt % an initiator.
wherein the material for enamels of posterior teeth have at least 10% difference in wear resistance than the material for the enamels of anterior teeth and the material for enamels of anterior teeth have at least 10% difference in fracture toughness than the material for enamels of posterior teeth.

2. The denture tooth system according to claim 1, wherein the material for enamels of anterior teeth include composition having:
a) about 35 to about 60% by weight liquid component including:
(i) about 60 to about 95 wt. % one or more monomers selected from the group of methyl methacrylate, methyl acrylate, ethyl methacrylate, isobutyl methacrylate, cyclohexylmethacrylate, isobornyl methacrylate, isobornyl acrylate, allyl methacrylate and mixtures thereof;
(ii) about 0 to about 15 wt. % a first crosslinking agents selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof;
(iii) about 0.5 to about 20 wt. % a second crosslinking agents selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof;
(iv) about 0.5 to about 15 wt. % of a third crosslinking agent selected from the group consisting of a reaction product of 1,3-bis(isocyanatomethyl)cyclohexane, 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane, 2-hydroxyethyl methacrylate, 2-hydroxy-3-phenoxypropyl acrylate, 2-hydroxyethyl acrylate; and mixtures thereof;
(v) about 0 to about 20 wt. % of a fourth crosslinking agent selected from the group consisting of a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof;
(vi) about 0 to about 10 wt % an initiator that includes a peroxide;
b) about 30 to about 65% by weight of particulate material.

3. The denture tooth system according to claim 1, wherein the material for enamels of posterior teeth include composition having:
a) about 35 to about 60% by weight liquid component including:
(i) about 40 to about 95 wt. % one or more monomers selected from the group of methyl methacrylate, methyl acrylate, ethyl methacrylate, isobutyl methacrylate, cyclohexylmethacrylate, isobornyl methacrylate, isobornyl acrylate, allyl methacrylate and mixtures thereof;
(ii) about 0 to about 15 wt. % a first crosslinking agents selected from the group of ethylene glycol dimethacrylate, glycerol di(meth)acrylate, glycerol tri(meth)acrylate, ethyleneglycol di(meth)acrylate, diethyleneglycol di(meth)acrylate, triethyleneglycol dimethacrylate, tetraethylene glycol di(meth)acrylate, 1,3-propanediol di(meth)acrylate, 1,3-propanediol dimethacrylate, trimethylolpropane tri(meth)acrylate, 1,2,4-butanetriol trimethacrylate, 1,4-cyclohexanediol diacrylate, 1,4-cyclohexanediol dimethacrylate, 1,6-hexanediol di(meth)acrylate, pentaerythritol tri(meth)acrylate, pentaerythritol tetra(meth)acrylate, pentaerythritol tetramethacrylate, sorbitol hexacrylate and mixtures thereof;
(iii) about 0 to about 20 wt. % a second crosslinking agents selected from the group consisting of 2,2-bis(4-methacryloxyphenyl)propane, 2,2-bis[4-(2-hydroxy-3-acryloyloxypropoxy)phenyl]propane, 2,2-bis[4-(2-hydroxy-3-methacryloyloxypropoxy)phenyl]propane, 2,2-bis[4-(acryloyloxy-ethoxy)phenyl]propane; 2,2-bis[4-(methacryloyloxy-ethoxy)phenyl]propane, and mixture thereof;
(iv) about 0 to about 40 wt. % of a third crosslinking agent selected from the group consisting of a reaction product of 1,3-bis(isocyanatomethyl)cyclohexane, 2-hydroxyethyl methacrylate, 2-hydroxy-3-phenoxypropyl acrylate, 2-hydroxyethyl acrylate and ethylene glycol dimethacrylate; a reaction product of 1,3-bis(isocyanatomethyl)cyclohexane, 2-hydroxyethyl methacrylate, 2-hydroxy-3-phenoxypropyl acrylate, 2-hydroxyethyl acrylate; and mixtures thereof;
(v) about 0 to about 20 wt. % of a fourth crosslinking agent selected from the group consisting of a reaction product of trimethyl 1,6-diisocyanatohexane and bisphenol A propoxylate and 2-hydroxyethyl methacrylate; a reaction product of 1,6 diisocyanatohexane and 2-hydroxyethyl methacrylate modified with water; a reaction product of 1,6 diisocyanatohexane and 2-hydroxyethyl acrylate modified with water; and mixtures thereof;
(vi) about 0 to about 10 wt % an initiator that includes a peroxide;
b) about 30 to about 65% by weight of particulate material including:
(i) a poly(methyl methacrylate) based component, and
(ii) a modified Poly(methyl methacrylate).

4. The denture tooth system according to claim 1, wherein the material for enamels of posterior teeth include composition having:
a) about 1 to about 80% by weight of liquid component including:
(i) about 40 to about 60 wt. % methyl methacrylate;
(ii) about 0 to about 15 wt. % ethylene glycol dimethacrylate;
(iii) about 10 to about 20 wt. % 2,2-bis(4-methacryloxyphenyl)propane;
(iv) about 15 to about 40 wt. % of a reaction product of 1,3-bis(isocyanatomethyl)cyclohexane, 2-hydroxyethyl methacrylate, 2-hydroxy-3-phenoxypropyl acrylate, 2-hydroxyethyl acrylate and ethylene glycol dimethacrylate,
(v) about 0 to about 10 wt. % of a reaction product of trimethyl 1,6-diisocyanatohexane and bisphenol A propoxylate and 2-hydroxyethyl methacrylate; and
(vi) about 0 to about 10 wt % benzoyl peroxide; and
b) about 30 to about 65% by weight of particulate material.

5. The denture tooth system according to claim 3, wherein the material for enamels of posterior teeth has a wear resistance ranging from about 0.035 to about 0.070 (Volume Loss: 37° C., mm³).

6. The denture tooth system according to claim 3, wherein the material for enamels of posterior teeth has a flexural strength ranging from about 135 to about 145 (MPa).

7. The denture tooth system according to claim 3, wherein the material for enamels of posterior teeth has a modulus ranging from about 3000 to about 3500 (MPa).

8. The denture tooth system according to claim 3, wherein the material for enamels of posterior teeth has a fracture toughness ranging from about 0.85 to about 1.85 (MPa m¹′²).

9. The denture tooth system according to claim 3, wherein the material for enamels of posterior teeth has a wear resistance ranging from about 0.035 to about 0.070 (Volume Loss: 37° C., mm³) and a flexural strength ranging from about 135 to about 145 (MPa).

10. The denture tooth system according to claim 3, wherein the material for enamels of posterior teeth has a modulus ranging from about 3000 to about 3500 and a fracture toughness ranging from about 0.85 to about 1.85 (MPa m^{1/2}).

11. The denture tooth system according to claim 2, wherein the material for enamels of anterior teeth has a fracture toughness ranging from about 1.8 to about 2.5 (MPa m^{1/2}).

12. The denture tooth system according to claim 2, wherein the material for enamels of anterior teeth has a wear resistance ranging from about 0.06 to about 0.125, (Volume Loss: 37° C., mm³).

13. The denture tooth system according to claim 2, wherein the material for enamels of anterior teeth has a fracture toughness ranging from about 1.8 to about 2.5 (MPa m^{1/2}) and a wear resistance ranging from about 0.06 to about 0.125, (Volume Loss: 37° C., mm³).
